(19) United States
(12) Patent Application Publication
(10) Pub. No.: US 2012/0172450 A1
(43) Pub. Date: Jul. 5, 2012

(54) USE OF JASMONATE FOR IMPROVING SKELETAL MUSCLE FUNCTION

(75) Inventor: Brunde Broady, New York, NY (US)

(73) Assignee: BROADY HEALTH SCIENCES, LLC, Coppell, TX (US)

(21) Appl. No.: 13/316,441

(22) Filed: Dec. 9, 2011

Related U.S. Application Data
(60) Provisional application No. 61/422,007, filed on Dec. 10, 2010.

(51) Int. Cl. A61K 31/122; A61K 31/185; A61P 21/00; A61K 31/198

(52) U.S. Cl. 514/565; 514/690; 514/578

(57) ABSTRACT

A formulation comprising jasmonate for improving skeletal muscle function in a mammal, particularly a human is provided. This may be accomplished by reducing muscle fatigue and/or increasing skeletal muscle performance.

### TECHNICAL FIELD

A formulation comprising jasmonate for improving skeletal muscle function in a mammal, particularly a human is provided. Skeletal muscle function may be improved by reducing muscle fatigue and/or increasing skeletal muscle performance.

### BACKGROUND

### Muscle Fatigue

When skeletal muscles are induced to contract repeatedly there is a progressive loss in their ability to generate a target force or power. This condition is known simply as fatigue. In particular, muscle fatigue may include, but is not limited to, muscle pain attributed to strenuous exercise, lassitude in the arms and legs resulting from acute exercise and lower back pain and stiffness in the shoulders due to holding a fixed posture over a prolonged period of time.

Several factors are known to participate in muscle fatigue. Examples include but are not limited to TCA cycle intermediates (see, for example, U.S. Pat. No. 6,143,784), amino acid metabolism such as deamination and protein catabolism (see U.S. Pat. No. 6,100,287), and decline in efficiency of muscle contraction. One factor in regulating efficiency of muscle contraction is the level of calcium in the sarcoplasmic reticulum (see, for example, Ortenblad et al., 2000, J. Appl. Physiol. 89:210-217). In muscle contraction, calcium is released from the sarcoplasmic reticulum into the cytoplasm, which triggers the contraction. Ca2+ATPase is responsible for refilling the sarcoplasmic reticulum with calcium after muscle contraction to effect muscle relaxation. Sarcoplasmic reticulum function, both Ca2+ release and Ca2+ uptake by Ca2+ATPase is impaired after fatiguing contractile activity (Tupling, 2004, Can J Appl Physiol 29:308-329; Li et al., 2002, J. Appl. Physiol. 92:912-922; Allen et al., 2008, J. Appl. Physiol 103:296-305). Studies show that acute high intensity exercise results in a decrease in Ca2+ATPase levels (Leppik et al., 2004, J. Appl. Physiol. 97:1414-1423; Steele et al., 2003, Acta Physiol. Scand. 179:39-48). As a result of the reduced Ca2+Atpase levels, calcium uptake into the sarcoplasmic reticulum is also reduced (William et al., 1997, J. Appl. Physiol. 83:444-450; Williams et al., 1998, J. Appl. Physiol. 85:619-626). Muscle fatigue may be age related, so that an elderly person may experience muscle fatigue with a minimal amount of movement and/or exercise.

Various substances have been proposed to treat muscle fatigue. Examples include, but are not limited to, glutamine peptide (see US Patent Application No. 2010/01648040), GAKIC, a formulation of glycine and L-arginine mono-hydrochloride salt of alpha-ketoisocaproic acid calcium (U.S. Pat. No. 6,100,287), pyruvate (U.S. Pat. No. 6,143,784), L-carnitine and optionally creatinol-phosphate (U.S. Pat. No. 6,602,512), reduced Coenzyme Q (US Patent Application Pub. No. 2006165672).

### Jasmonates

Jasmonates are a family of plant stress hormones that are found in minute quantities in edible plants and characterized by cyclopentone rings. Various uses for jasmonates have been disclosed. Examples include enhancing plant growth (U.S. Pat. No. 5,436,226), repelling insects (U.S. Pat. No. 5,118,711), treating cancer (U.S. Pat. No. 6,469,061) and treating skeletal muscle degeneration caused by malnutrition and disease (U.S. Pat. No. 6,465,021, US Patent Appln. Pub. No. 201000003346), pain relief (WO 2009019693), relieving psychological stress (US Patent Appln. Pub. No. 200700420567), use as a component of a sleep supplement (JP2000355545), treating dry skin (US Patent Appln. Pub. No. 20110085999), treating malodors on fabrics (US Patent Appln. Pub. No. 20110070181) treating heart failure and related disorders (US2011. Jasmonate has also been found to increase Ca2+Atpase in cardiac sarcoplasmic reticulum (see, for example, Antipenko et al., 1997, J. Biol. Chem. 272:2852-60) and skeletal muscle (see, for example, Ioumaa et al., 2002, J. Pharmacol. Exp. Ther. 300:638-46; Starling et al, 1995, Biochem. J. 308:343-6 and Starling et al., 1994, Biochemistry 15:3023-31).

### SUMMARY

Disclosed herein is a method for reducing skeletal muscle fatigue and/or increasing skeletal muscle performance in a mammal, particularly a human, in need thereof comprising administering an amount of a at least one jasmonate and optionally at least one other substance, wherein said substance is a drug or natural substance used to reduce muscle fatigue and/or increasing muscle performance, effective to modulate sarcoplasmic reticulum function in the skeletal muscle of said mammal. In a related aspect, the invention relates to a method for reducing skeletal muscle fatigue and/or increasing skeletal muscle performance in a mammal in need thereof, comprising administering an amount of at least one jasmonate effective to reduce muscle fatigue and/or increase skeletal muscle performance.

In a further aspect, also provided is a composition which may be used in reducing muscle fatigue, increasing sarcoplasmic reticulum function in the skeletal muscle and/or increasing skeletal muscle performance in a mammal in need thereof comprising jasmonate. The composition may further comprise at least one other substance, wherein said substance is a drug or natural substance used to reduce muscle fatigue and/or increase muscle performance. This substance may be an antioxidant, a vitamin, amino acid and/or nutritional supplement. The composition may comprise jasmonate (e.g., methyl jasmonate), at least two basic amino acids (e.g., arginine and citrulline) and a nutritional supplement (e.g. taurine). The composition may comprise jasmonate, arginine, citrulline and taurine. In particular, the composition may comprise methyl jasmonate, arginine, citrulline and taurine or alternatively, cis-jasmone, arginine, citrulline and taurine.

In a related aspect, also disclosed is the use of jasmonate and optionally at least one other substance, wherein said substance is a drug or natural substance used to reduce muscle fatigue and/or increase skeletal muscle performance and/or increase sarcoplasmic reticulum function in the skeletal muscle of said mammal for formulating a medicament for use in reducing muscle fatigue and/or increasing skeletal muscle performance and/or increasing sarcoplasmic reticulum function in the skeletal muscle of said mammal.

### DETAILED DESCRIPTION OF SPECIFIC EMBODIMENTS

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Definitions

As defined herein, the term “modulate” means adjusting the sarcoplasmic endoplasmic reticulum function by, preferably increasing Ca2+ ATPase levels and/or amount and/or rate of Ca2+ release.

As defined herein, the term “skeletal muscle fatigue” occurs when these muscles cannot exert normal force, or when more effort than normal is required to achieve a desired level of force. Muscle fatigue may include but is not limited to exercise-induced muscle fatigue, posture induced muscle fatigue. Exercise-induced muscle fatigue may occur during and after exercise. Muscle fatigue may be decreased by retarding the onset and/or duration of muscle fatigue.

As defined herein, the term “increasing skeletal muscle performance” means, for example, increasing muscle strength, increasing muscle endurance and in particular increasing the ability to sustain athletic muscle force and/or work during muscle exercise.

### Jasmonates

The jasmonates used in the compositions and methods of the present invention may have the formula I

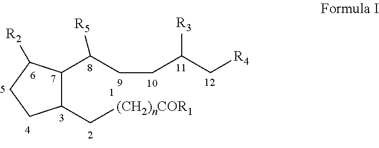

wherein:

n is 0, 1, or 2;

R₁ is OH, alkoxy, O-glucosyl, or imino,

R₂ is OH, O, alkoxy, or O-glucosyl,

R₃, R₄, and R₅ are H, OH, alkoxy or O-glucosyl,

and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following:

a lower acyl side chain at C₃ (free acid or ester or conjugate), a keto or hydroxy (free hydroxy or ester) moiety at the C₆ carbon, or an n-pentenyl or n-pentyl side chain at C₇.

In a particular embodiment, the jasmonate may be selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-β-glucopyranoside, cucurbic acid-O-β-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

### COMPOSITIONS

The compositions may comprise the jasmonate set forth above. Additionally, the compositions may further comprise least one other drug or natural substance used to reduce muscle fatigue and/or improve muscle performance. This drug or substance may include but is not limited to an antioxidant or an antioxidant enzyme. Antioxidants include but are not limited to vitamin E, vitamin E derivatives, vitamin C, vitamin C derivatives, probucol, lycopene, vitamin A, carotenoids, vitamin B, vitamin B derivatives, flavonoids, polyphenols, glutathione, pyrroloquinoline quinone, pycnogenol, flavangenol, or selenium may be suitable. The antioxidants described above may be used singly or as a mixture of the two or more. The antioxidant enzyme is not limited in particular, but, for example, superoxide dismutase (SOD), glutathione peroxidase, glutathione-S-transferase, glutathione reductase, catalase or ascorbic acid peroxidase may be suitable. The antioxidant enzymes described above may be used singly or as a mixture of the two or more.

The other substance may also be other nutrient and tonic components. The nutrient and tonic components are not limited in particular, but, for example, creatine, taurine, vitamin B1, vitamin B derivatives, or amino acids may be suitable. The nutrients and tonics described above may be used singly or as a mixture of the two or more. An additive or a synergetic effect may be expected by mixing jasmonate with these components.

The other substance may also contain nutritional supplements. The nutritional supplements are not limited in particular, but include amino acids, metal ions, sugars, proteins, fatty acids, vitamins and the like.

The compositions may also include jasmonates in combination with a drug or substance that may include, but is not limited to, Astragalus or substances derived therefrom (e.g., astragaloside), gingerol, green tea or substances derived therefrom (e.g., epigallocatechin gallate), gingerol.

In a particular embodiment, the composition may comprise jasmonate (e.g., methyl jasmonate) and one or more amino acids as active ingedients. These amino acids may include but is not limited to taurine, arginine and citrulline.

The compositions, in particular, pharmaceutical compositions of the present invention can be formulated for administration by a variety of routes including oral, transdermal, parenteral (subcutaneous, intraperitoneal, intravenous, intra-arterial, and intramuscular) or rectal. Such compositions are prepared in a manner well known in the pharmaceutical art and comprise as an active ingredient at least one of the compounds used in the method of the present invention as described herein above ad a pharmaceutically acceptable excipients or a carrier. The amount of the active ingredient(s) in the composition of the present invention is from about 0.5 to 100% per weight. The term “pharmaceutically acceptable” means approved by a regulatory agency of the Federal or a state government or listed in the U.S. Pharmacopeia or other generally recognized pharmacopeia for use in animals and, more particularly, in humans. Choosing suitable ingredients for the composition is a routine for those of ordinary skill in the art. It is evident that suitable carriers, solvents, gel forming ingredients, dispersion forming ingredients, antioxidants, colors, sweeteners, wetting compounds, release controlling components and other ingredients normally used in this field of technology may be also used. Pharmaceutical compositions in the form of intravenous solutions are preferred. The active ingredients may be formulated in the same pharmaceutical formulation. Alternatively, the active ingredients are formulated as separate pharmaceutical dosage forms. The combination of the pharmaceutical dosage forms may be packaged as a single medical product or kit for use in the method of the invention, optionally together with a package insert instructing to the correct use of the medical product.

Another preferred embodiment of the invention is a medical product comprising, separately or together, as active ingredients one or more jasmonates or a pharmaceutically acceptable salt thereof and one or more other substance used for reducing muscle fatigue and/or increasing skeletal muscle performance in a mammal as a combined preparation.

In yet another embodiment, the medical product may comprise one two or more jasmonates set forth above in separate compositions.

### ADMINISTRATION AND USES

As noted above, one or more jasmonate(s) optionally in combination with other substances may be used to reduce muscle fatigue and/or increase skeletal muscle performance in a mammal, e.g., a human. Muscle fatigue may be due to exercise-related muscle fatigue or posture related muscle fatigue.

The active ingredients may be administered simultaneously, separately or sequentially. The administration routes of the active ingredients include, but are not limited to, enteral, e.g. oral or rectal, or parenteral, e.g. intravenous, intramuscular, topical, intraperitoneal or transdermal. The formulation is not limited in particular when the agent is applied directly to the skin but, for example, the following formulations may be used: a cream, a paste, a jelly, a gel, an emulsion or a liquid formulation (an ointment, a liniment, a lotion, a cream, a spray and the like) in which the agent substances described above are dissolved, or mixed and dispersed in a suitable base; a formulation in which the agent substances are dissolved, or mixed and dispersed in a base, and spread over a support medium (a poultice and the like); a formulation in which the agent substances described above are dissolved, or mixed and dispersed in a sticker and spread over a support medium (a plaster, a tape and the like) The bases and the stickers, which are normally used in pharmaceuticals and cosmetics, may be used at need as long as the efficacy of the present invention is not impaired.

The compositions used may preferably be administered orally preferably once per day. According to the invention, the suggested daily dose of jasmonate(s) is in general from about 0.01 to 50 mg, preferably from about 0.02 to 20 mg, more preferably from about 0.05 to 10 mg, and even more preferably from about 0.05 mg-0.10 mg, depending on the age, body weight and condition of the patient. The effective amount of jasmonate(s) to be administered to a subject depends upon the condition to be treated, the route of administration, age, weight and the condition of the patient. Similar dosages of other substances may also be used.

### EXAMPLES

The composition and methods set forth above will be further illustrated in the following, non-limiting Examples. The examples are illustrative of various embodiments only and do not limit the claimed invention regarding the materials, conditions, weight ratios, process parameters and the like recited herein.

### Effect of Jasmone on Muscle Fatigue and Performance

Subjects ranged from 43 to 72 years old. Subjects took 50-100 micrograms of jasmone once a day. All subjects were currently performing various exercise programs. Individual data was collected as to the changes that were noted upon jasmone supplementation.

Subject one, was a 72 year old male who ran and trained with weights on a regular basis. He took a simplified stress test measuring time to maximum heart rate on treadmill exertion. Pre-Jasmone time on treadmill to target heart rate was 9 minutes. Two weeks after taking Jasmone, stress test results improved to 11 minutes. After 4 weeks of jasmone protocol stress test time increased to 15 min. In addition, after 3 weeks he reported increased weight repetitions by 15 percent and within four weeks increased push ups by 20 percent with same level of exertion as before.

Subject two, was a 55 yr. old man who ran on a regular basis. Prior to Jasmone, full exertion exercise routine consisted of approximately 2 miles at 4.5 miles per hour. After two weeks of Jasmone was able to increase routine to running time to 2.5 miles without difficulty. After additional two weeks workout fatigue continued to improve and additional components, i.e. sprint sessions were easily incorporated.

Subject three, was a 50 year old woman who biked on a regular basis. After 4 weeks of taking Jasmone she increased cycling distance by 50 percent and speed by 10 percent.

Subject four, a 68 year old man reported who trained with weights on a regular basis, increased weight repetitions of 25% after 4 weeks of taking Jasmone. Subject four, a 73 year old man regularly performed an exercise routine including weight machines. Prior to taking Jasmone, on a particular machine, he was able to perform 12 repetitions at 60 pds of weight. Two weeks after taking Jasmone was able to increase weight to 80 and repetitions increased to 14. After four weeks of Jasmone, he felt increased strength and endurance and found routine progressively easier.

Subject five, was a 43 year old man, who regularly exercised but due to travel was unable to maintain normal exercise routine during trial. However he did report increased ability to run far and fast without getting winded. Two examples of this include running through an airport carrying luggage and another running in extreme heat.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

What is claimed is:

1. A method of reducing muscle fatigue and/or increasing skeletal muscle performance in a mammal in need thereof, the method comprising administering to said mammal, an amount of jasmonate effective to increase sarcoplasmic reticulum function in the skeletal muscle of said mammal and/or reduce muscle fatigue of said mammal and/or increase skeletal muscle performance of said mammal.

2. The method according to claim 2, wherein said muscle fatigue is exercise-induced muscle fatigue or posture-induced muscle fatigue.

3. The method according to claim 1, wherein said wherein said jasmonate has the structure (I) 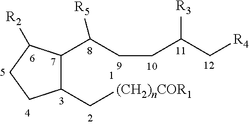
wherein:
n is 0, 1, or 2;
R₁ is OH, alkoxy, O-glucosyl, or imino,
R₂ is OH, O, alkoxy, or O-glucosyl,
R₃, R₄, and R₅ are H, OH, alkoxy or O-glucosyl,
and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following: a lower acyl side chain at C₃ (free acid or ester or conjugate), a keto or hydroxy (free hydroxy or ester) moiety at the C₆ carbon, or an n-pentenyl or n-pentyl side chain at C₇.

4. The method according to claim 3, wherein said jasmonate is a compound selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-β-glucopyranoside, cucurbic acid-O-β-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

5. The method according to claim 1, which further administering at least one other substance, wherein said substance is a drug or natural substance used to reduce muscle fatigue and/or increase muscle performance.

6. The method to claim 5, wherein said substance is an antioxidant, a vitamin, amino acid and/or nutritional supplement.

7. The method according to claim 5, wherein jasmonate, citrulline, argininine and taurine is administered to said mammal.

8. A composition which comprises jasmonate, taurine, and at least two basic amino acids.
